(19) United States
(12) Patent Application Publication
(10) Pub. No.: US 2023/0386612 A1
(43) Pub. Date: Nov. 30, 2023

(54) DETERMINING COMPARABLE PATIENTS ON THE BASIS OF ONTOLOGIES

(71) Applicant: Siemens Healthcare GmbH, Erlangen (DE)

(72) Inventors: Oliver FRINGS, Erlangen (DE); Carsten DIETRICH, Nuernberg (DE); Maximilian WEISS, Nuernberg (DE); Matthias SIEBERT, Marloffstein (DE); Mitchell JOBLIN, Surrey (CA)

(73) Assignee: Siemens Healthcare GmbH, Erlangen (DE)

(21) Appl. No.: 18/246,731

(22) PCT Filed: Sep. 7, 2021
(86) PCT No.: PCT/EP2021/074567

§ 371 Date: Mar. 27, 2023

(30) Foreign Application Priority Data
Sep. 30, 2020 (DE) 10 2020 212 379.9

(51) Int. Cl. G16B 50/10; G16H 20/00; G16H 50/70; G16H 10/60

(52) U.S. Cl. CPC G16B 50/10; G16H 20/00; G16H 50/70; G16H 10/60

(57) ABSTRACT

One or more example embodiments relates to a computer-implemented method for determining a similarity measure, the similarity measure describing a similarity between a first patient and a second patient. The method includes receiving a first patient data record, wherein the first patient data record is assigned to the first patient; receiving a second patient data record, wherein the second patient data record is assigned to the second patient; receiving or determining a medical ontology, wherein the medical ontology is independent of the first patient data record and the second patient data record; determining a patient ontology based on the medical ontology and at least one of the first patient data record or the second patient data record; and determining the similarity measure based on the patient ontology.

### PRIORITY STATEMENT

This application is the national phase under 35 U.S.C. § 371 of PCT International Application No. PCT/EP2021/074567 which has an International filing date of Sep. 7, 2021, which claims priority to German Application No. 10 2020 212 379.9, filed on Sep. 30, 2020, which designated the United States of America, the entire contents of each of which are hereby incorporated herein by reference.

### RELATED ART

Systems biology or bioinformatics, in particular the subfields of genomics, transcriptomics or proteomics, are a rapidly developing field of medical research. In some cases, clinical guidelines are unable to follow this rapid development (and therefore no longer represent clinical practice), or sometimes do not take this information into account at all. This makes it difficult for medical professionals to find the best treatment alternative for a patient, in particular for cancer patients.

Cancer therapies are increasingly targeted at specific molecular genetic abnormalities in cancer cells (so-called somatic mutations). Such cancer therapies are generally only approved for a specific form of cancer (for example, an affected tissue) and a specific somatic mutation. It is generally unclear whether and, if so, which additional patients can benefit from such treatment. Furthermore, other genetic mutations in a patient may also influence the outcome of treatment.

A common approach for selecting a treatment alternative for a patient is to compare the patient with other patients who are clinically similar in order to assess the outcome of treatment alternatives. However, there is no universally accepted definition of clinical or molecular genetic similarity, and, furthermore, the determination of molecular genetic similarity is challenging in itself. Therefore, it is common for patients to be compared based on the presence or absence of somatic mutations. However, such a comparison is only an imprecise approximation of the patient's phenotype. For example, a somatic mutation does not necessarily imply that the affected gene is also expressed in the cancer cells.

The shortcomings of the usual method are particularly apparent in the case of cancer therapy because cancer is generally a highly complex condition in which certain cells of the human body have acquired the ability to divide and reproduce in an uncontrolled manner. Somatic or epigenetic changes in individual cells are one cause of this behavior because they influence important processes in human cells, for example, the cell cycle, apoptosis or cell growth. However, in addition, the processes in the cells are also influenced by a complex interplay of multiple genes and the proteins they encode, which are described by signaling pathways and regulatory pathways.

If only genetic mutations are considered when searching for similar patients, the influences of these complex interactions are neglected.

### SUMMARY

It is therefore the object of the present invention to improve the selection of comparable patients. This object is achieved by a computer-implemented method for determining a similarity measure, by a determination system, by a computer program product and by a computer-readable storage medium as claimed in the independent claims. Advantageous embodiments and developments are presented in the dependent claims and in the following description.

The way in which the object is achieved according to the embodiments of the invention is described below with respect to both the claimed apparatuses and the claimed method. Features, advantages or alternative embodiments mentioned herein are equally applicable to other claimed subject matter and vice versa. In other words, the substantive claims (directed, for example, at an apparatus) can also be developed with the features described or claimed in connection with a method. Herein, the corresponding functional features of the method are embodied by corresponding substantive modules.

In a first aspect, one or more embodiments of the invention relates to a computer-implemented method for determining a similarity measure, wherein the similarity measure describes a similarity between a first patient and a second patient. The method is based on the reception of a first patient data record and a second patient data record, wherein the first patient data record is assigned to the first patient, and wherein the second patient data record is assigned to the second patient. Furthermore, a medical ontology is received or determined. In this case, the medical ontology is independent of the first patient data record and the second patient data record. Furthermore, a patient ontology is determined based on the medical ontology and furthermore based on the first patient data record and/or the second patient data record. Furthermore, a similarity measure is determined based on the patient ontology. Optionally, furthermore, the similarity measure is provided, wherein the provision can comprise storing, transmitting and/or representing the similarity measure.

The first patient data record and the second patient data record can in particular be received via an interface, in particular via an interface of a determination system. The medical ontology can in particular be received or determined via the interface or a computing unit, in particular via the interface of the determination system or a computing unit of the determination system. The patient ontology can in particular be determined via the computing unit, in particular via the computing unit of the determination system. The similarity measure can in particular be determined via the computing unit, in particular via the computing unit of the determination system.

A patient data record comprises medical data from a patient and is in particular assigned to the patient whose data it comprises. A patient data record is in particular assigned to exactly one patient.

A patient data record can in particular comprise genetic information about a patient, for example, a gene sequence. A patient data record can in particular also comprise the data relating to the data from an HIS (hospital information system), an RIS (radiology information system), a LIS (laboratory information system,) or a PACS (picture archiving and communication system. A patient data record can in particular be identical to an EMR (electronic medical record) of the patient, the entire EMR or parts of the EMR.

An ontology is in particular a formally ordered representation of a network of concepts, data and/or information and the relationships between them in a specific field. An ontology can in particular be used to exchange information in digitized and formal form between application programs and services. In particular, an ontology represents a network of concepts, data and/or information that are logically related. An ontology can in particular contain rules of inference and integrity, i.e., rules for drawing conclusions and for ensuring their validity. An ontology can in particular be represented in the form of a mathematical graph comprising nodes and edges, in particular in the form of a directed graph. In this case, the nodes and/or the edges can in particular include further data.

The term ontology is sometimes used for both the definition of a schema or a class (sometimes referred to as an ontology template) and for the associated instance or the associated object. The term “ontology” can be used in both meanings within this document, but, in case of doubt, “ontology” is used in each case as a term for an instance or implementation of an abstract schema.

A medical ontology is in particular an ontology relating to medical and/or (human) biological facts. In this case, a medical ontology is in particular independent of the specific patient, in other words, a medical ontology represents and structures existing abstract technical or domain knowledge. Such a medical ontology can be the result of scientific research, in particular with regard to causal relationships and the structure of medical information.

A medical ontology is in particular not based on the first patient data record and/or the second patient data record. In other words, therefore, the medical ontology does not comprise any information about the first patient and/or the second patient.

Examples of medical ontology are known systems biology relationships (in particular interactions between human genomes, epigenomes, transcriptomes, proteomes and/or metabolomes, and their phenotypic effects) and classification systems for symptoms and/or diseases, such as, for example, the International Statistical Classification of Diseases and Related Health Problems (ICD) in the ninth or tenth version (ICD-9 or ICD-10), the International Classification of Functioning, Disability and Health (ICF), the Medical Subject Headings (MeSH), the Systematized Nomenclature of Medicine (SNOMED) or the Unified Medical Language System (UMLS).

The medical ontology can be received or determined in the method according to the aspect of one or more embodiments of the invention. The determination of medical ontology can in particular comprise the determination of a corresponding graph based on structured and/or unstructured medical information. For example, a graph structure can be extracted from a text document.

A patient ontology in particular describes the combination of a medical ontology and data relating to a specific patient. In particular, therefore, a patient data record is integrated into a medical ontology, or the patient data record and the medical ontology are combined.

A patient ontology can in particular be concretized via the data from exactly one patient, or, in other words, comprise data from exactly one patient. Alternatively, a patient ontology can also be concretized via the data from a plurality of patients, or, in other words, comprise data from a plurality of patients.

To concretize a medical ontology into a patient ontology, it is in particular possible to adapt elements of the medical ontology based on the patient data. Alternatively, additional elements can be added to the medical ontology based on the patient data.

A similarity measure is in particular a numerical value, in particular a real number between 0 and 1, inclusive in each case. In particular, the similarity measure can also be a binary value, in particular “1” or “true”, if the first patient and the second patient are similar, and “0” or “false”, if the first patient and the second patient are not similar. A similarity measure can in particular also comprise a plurality of real numbers (in particular in the form of a vector), each of the real numbers can in particular in turn assume a value between 0 and 1 and/or be a binary value. In particular, therefore, the similarity measure maps at least the patient ontology to a number and/or a vector. The similarity measure can also be used for a comparison between different second patients, for example, a ranking of the second patients (with increasing or decreasing similarity to the first patient).

The inventors have recognized that the use of a patient ontology enables the patient data to be structured very efficiently and to be linked to pre-existing knowledge about medical and/or (human) biological relationships. In particular, it is also possible for relationships between individual data points in the patient data to be mapped or also relationships between a plurality of patients and a large number of individual and different data points to be acquired for one or more patients. This in particular also enables similarity measures for patients to be determined reliably based on heterogeneous data.

According to a further aspect of one or more embodiments of the invention, the patient ontology is a common patient ontology, wherein the common patient ontology is based on the medical ontology, the first patient data record and the second patient data record. In other words, the common patient ontology concretizes the medical ontology via the first patient data record and the second patient data record.

One or more embodiments of the invention according to this aspect can in particular relate to a computer-implemented method for determining a similarity measure, wherein the similarity measure describes a similarity between a first patient and a second patient, comprising: receiving a first patient data record, wherein the first patient data record is assigned to the first patient; receiving a second patient data record, wherein the second patient data record is assigned to the second patient; receiving or determining a medical ontology, wherein the medical ontology is independent of the first patient data record and the second patient data record; determining a common patient ontology based on the medical ontology, the first patient data record and the second patient data record; and determining the similarity measure based on the common patient ontology.

It is in particular possible for the common patient ontology to be based on a plurality of second patient data records. In other words, it is possible for the common patient ontology to concretize the medical ontology based on the data from the first patient and a plurality of second patients.

The inventors have recognized that the use of a common patient ontology comprising the data from a plurality of patients means there is no need to duplicate the patient-independent knowledge. Therefore, compared to a plurality of separate patient ontologies, a common patient ontology is less memory intensive and can be transferred more quickly.

According to a further aspect of one or more embodiments of the invention, the common patient ontology comprises a graph, wherein a subgraph relates to the medical ontology. In this case, the first patient data relates to at least one first node of the graph outside the subgraph and at least one edge between the first node and the subgraph. The second patient data furthermore relates to at least one second node of the graph outside the subgraph and at least one edge between the second node and the subgraph. In this aspect, the similarity measure is based on a probability of an edge between the first node and the second node. In particular, here, the first node and the second node are different nodes.

A graph is an abstract structure representing a set of objects together with connections between these objects. A representative of an object is called a node, a representative of a connection is called an edge. In particular, an edge is assigned to at most two nodes, wherein the edge then connects these nodes.

A directed graph is a graph in which the edges have an orientation. In particular, therefore, an edge has a first node as its beginning and a second node as its end (and differs from an edge that has the second node as its beginning and the first node as its end). In this case, the first and the second node can be different or identical nodes. An undirected graph is a graph in which the edges have no orientation. In particular, here, an edge can be defined as a set of two nodes. It is possible to use mixed graphs comprising both directed and undirected edges.

A first graph is a subgraph of a second graph if, by deleting nodes, the edges belonging to the deleted nodes and any possible further edges, the second graph can be transformed into the first graph. The first graph is also a subgraph of a second graph if the first graph and the second graph are identical. Non-identical subgraphs can also be called proper subgraphs. An edge between a node and a subgraph is a (directed or undirected) edge between the node and a node of the subgraph.

To calculate the probability of an edge between two nodes (also called link prediction or graph completion), it is, in particular, assumed that a present graph is a subgraph of an unknown graph, wherein the unknown graph in particular has the same edges as the present graph. In this case, the one edge between two nodes (in the present graph) in particular corresponds to the probability that there is an edge between the corresponding nodes in the unknown graph. The similarity measure can in particular be identical to the probability of the edge, but alternatively the similarity measure can also be based on further data from the common patient ontology, for example, node data.

Known methods for determining the probability are in particular topological methods, node-attribute-based methods and combinations of these two methods.

The inventors have recognized that the structure of ontologies can be described particularly well by directed and/or undirected graphs. In this case, the edges in particular correspond to the relations in the respective ontology. The use of a similarity measure based on the graphs in particular enables the structure of the ontology to be used. Such a determination is more resource-efficient and can make use of known measures and graph theory algorithms.

According to a further aspect of one or more embodiments of the invention, the method comprises determining a first patient ontology based on the common patient ontology and determining a second patient ontology based on the common patient ontology. In this case, the determination of the similarity measure is based on the first patient ontology and the second patient ontology. In particular, the determination of the similarity measure is not based on the common patient ontology or the determination of the similarity measure is only based on the common patient ontology to the extent that the determination of the similarity measure is based on the first and second patient ontology derived from the common patient ontology.

The inventors have recognized that the use of the first and the second patient ontology enables them to be stored and transmitted separately. This enables a decentralized comparison (including with other patient ontologies); this is in particular advantageous from the point of view of data protection.

According to a further aspect of one or more embodiments of the invention, the patient ontology is a first patient ontology, wherein the first patient ontology is not based on the second patient data record. The method furthermore comprises determining a second patient ontology based on the medical ontology and the second patient data record. In particular, the second patient ontology is not based on the first patient data record. In this case, the determination of the similarity measure is based on the first patient ontology and the second patient ontology.

According to this aspect, one or more embodiments of the invention can in particular relate to a computer-implemented method for determining a similarity measure, wherein the similarity measure describes a similarity between a first patient and a second patient, comprising: receiving a first patient data record, wherein the first patient data record is assigned to the first patient; receiving a second patient data record, wherein the second patient data record is assigned to the second patient; receiving or determining a medical ontology, wherein the medical ontology is independent of the first patient data record and the second patient data record; determining a first patient ontology based on the medical ontology and the first patient data record; determining a second patient ontology based on the medical ontology and the second patient data record; and determining the similarity measure based on the first patient ontology and the second patient ontology.

The inventors have recognized that the use of the first and the second patient ontology enables them to be stored and transmitted separately. This enables decentralized comparison (including with other patient ontologies); this is in particular advantageous from the point of view of data protection. At the same time, no separate common patient ontology needs to be determined in this aspect.

According to a further aspect of one or more embodiments of the invention, the first patient ontology and the second patient ontology in each case comprise a graph. In this case, the similarity measure is based on a similarity between the first graph of the first patient ontology and the second graph of the second patient ontology. In particular, here, the graphs are directed graphs.

The similarity measure can in particular be identical to the similarity of the first and the second graph, but alternatively the similarity measure can also be based on further data from the common patient ontology, for example, node data.

The inventors have recognized that the structure of ontologies can be described particularly well by graphs. In this case, the directed edges in particular correspond to the relations in the respective ontology. The use of a similarity measure based on the graphs enables the similarity to be determined based on the structure of the ontology. Such a determination is resource-efficient and can make use of known measures and graph theory algorithms.

According to a further aspect of one or more embodiments of the invention, the similarity measure comprises the graph edit distance of the first graph and the second graph and/or the maximum common subgraph distance of the first graph and the second graph.

The graph edit distance in particular measures a minimal number of elementary changes for transforming a first graph into a second graph. In particular, a weighting can be assigned to an elementary change and the graph edit distance is then the minimum weighted number of changes in order to transform a first graph into a second graph. Elementary changes are in particular the insertion or deletion of nodes or edges; sometimes the operations of edge splitting (inserting a node into an edge, thereby replacing the edge by this node and two edges coincident with the node edges) and edge merging (deleting a binary node and the two associated edges and replacement by an edge) are called elementary changes.

A maximum common subgraph distance is a similarity measure between two graphs based on a maximum common subgraph. A maximum common subgraph is, for example, the subgraph of a first and a second graph with the largest number of edges or the largest number of nodes. The similarity measure based on such a maximum common subgraph can be defined by this maximum number of nodes or edges or by a ratio based on this number of nodes or edges and the number of nodes or edges of the first and/or the second graph.

The inventors have recognized that, based on the graph edit distance or the maximum common subgraph distance, the comparison of the ontologies can be performed in a fault-tolerant manner, and, as a result, missing data or faulty data has less influence on the comparison of the ontologies.

According to a further aspect of one or more embodiments of the invention, the similarity measure is based on vertex embedding and/or graph embedding of the first directed graph and the second directed graph.

Vertex embedding of a graph in particular maps each node of a graph into a vector space, in particular into a n-dimensional real vector space. In other words, therefore, each node is coordinated or each node is assigned coordinates.

Graph embedding of a graph in particular maps the entire graph onto a vector. For example, an adjacency matrix can be interpreted as graph embedding. However, in particular, graph embedding uses a vector with a smaller dimension than the adjacency matrix of the graph.

Various methods are known for determining graph embeddings. Known algorithms for determining vertex embedding are DeepWalk and Node2vec (based on random walks), and structural deep network embedding based on artificial neural networks embodied as autoencoders. A known algorithm for determining graph embedding is Graph2vec. An overview of known methods can be found in the article: H. Cai: A Comprehensive Survey of Graph Embedding: Problems, Techniques and Applications, arXiv:1709.07604v3 (2017) and in the article: P. Cui: A Survey on Network Embedding, arXiv:1711.08752v1 (2017); the methods and algorithms mentioned there can be used according to various exemplary embodiments of one or more embodiments of the invention for determining vertex embedding and/or graph embedding.

A similarity measure based on vertex embedding can in particular be based on the distance between nodes embedded in the vector space, i.e., in particular on a metric or norm in this vector space. A similarity measure based on graph embedding can in particular be based on a distance in the vector space of the embedded graphs.

The inventors have recognized that, for similarity measures based on graph embedding, it is possible to use known methods and algorithms in vector spaces that are not defined or not available on graphs. In particular, computing operations in vector spaces are usually faster and more efficient than corresponding computing operations in graphs. In particular, computing operations in vector spaces can be parallelized more easily than the corresponding computing operations on graphs.

According to a further aspect of one or more embodiments of the invention, the calculation of the similarity measure is based on an application of a trained function to the first patient ontology and the second patient ontology.

In particular, the first patient ontology and the second patient ontology are used as input data for the trained function in order to use the similarity measure as output data of the trained function.

A trained function maps input data to output data. In this case, the output data can in particular continue to depend on one or more parameters of the trained function. The one or more parameters of the trained function can be determined and/or adapted by training. For example, the parameter can be adapted by supervised learning, semi-supervised learning and/or by unsupervised learning.

The determination and/or adaptation of the one or more parameters of the trained function can in particular be based on a pair consisting of training input data and associated training output data, wherein the trained function is applied to the training input data to generate training mapping data. In particular, the determination and/or the adaptation can be based on a comparison of the training mapping data and the training output data. In general, a trainable function, i.e., a function with one or more parameters that have not yet been adapted, is also referred to as a trained function.

Other terms for trained function are trained mapping rule, mapping rule with trained parameters, function with trained parameters, algorithm based on artificial intelligence, machine learning algorithm. An example of a trained function is an artificial neural network, wherein the edge weights of the artificial neural network correspond to the parameters of the trained function. Instead of the term neural network, it is also possible to use the term neural net. In particular, a trained function can also be a deep neural network or deep artificial neural network. A further example of a trained function is a support vector machine; it is further also possible to use in particular other machine learning algorithms as the trained function.

Patient ontologies can in particular be used as input data for a trained function in that the patient ontology is interpreted and/or represented as a directed or undirected graph and the input data for the trained function in each case comprises the adjacency matrix. The input data can, of course, comprise further values, in particular values that are assigned to the respective nodes and/or edges (in particular coordinates of vertices) in a graph representation of a patient ontology. During training, in each case a pair of patient ontologies can be assigned a similarity measure that serves as a ground truth in supervised learning.

Applications of neural networks as a trained function for the comparison of directed and undirected graphs are also known from the document: Yunsheng Bai et al., SimGNN: A Neural Network Approach to Fast Graph Similarity Computation, WSDM'19 (2019) https://doi.org/10.1145/3289600.3290967. Furthermore, in particular graph neural networks, graph convolutional networks and/or graph recurrent neural networks can be used. Further methods are known from the document: Z. Zhang et al., Deep Learning on Graphs: A Survey, arXiv:1812.04202v1 (2018).

The inventors have recognized that the use of a trained function for determining similarity measures enables hidden and/or unknown relationships in the patient ontologies to be used for the determination of the similarity measure since the trained function is able to evaluate and exploit such relationships or correlations. Furthermore, the application of a trained function, in particular a neural network, after the training phase is very resource-efficient.

According to a further aspect, the similarity measure is determined for a plurality of second patient data records, wherein the second patient data records are assigned to a plurality of second patients. In this case, the method furthermore comprises determining a set of comparable patients based on the determined similarity measures, wherein the set of comparable patients is a subset of the plurality of second patients, and wherein in particular each of the comparable patients is similar to the first patient.

In other words, therefore, the method is executed for each of the second patients or for each of the second patient data records and a similarity measure is determined for each of the second patients. The set of comparable patients is then performed based on certain similarity measures.

The plurality of second patients comprises at least two second patients. The first patient can be part of the plurality of second patients, however, advantageously, the first patient is not part of the plurality of second patients.

The set of comparable patients is a subset of the plurality of second patients. In particular, therefore, each comparable patient is also contained in the plurality of second patients, but, at the same time, not each of the second patients is necessarily contained in the set of comparable patients. The set of comparable patients and the plurality of second patients can be identical, but advantageously the set of comparable patients is a true subset of the plurality of second patients, i.e., at least one of the second patients is not contained in the set of comparable patients. The set of comparable patients can in particular also comprise exactly one comparable patient. The set of comparable patients can also be an empty set.

The set of second patient data records in particular has the same power as the set of second patients. In particular, therefore, a patient data record from the set of second patient data records is uniquely assigned to each patient. The patient data record uniquely assigned to a patient in particular comprises data from this patient.

In this case, the similarity measure can also be used for a comparison or ranking between the plurality of second patients, for example, ranking of the second patients with increasing or decreasing similarity to the first patient.

According to a further possible aspect of one or more embodiments of the invention, the set of comparable patients is determined based on a comparison of the respective similarity measure with a threshold value. In particular, all second patients for whom the calculated similarity measure is above the threshold value are assigned to the comparable patients and all second patients for whom the calculated similarity measure is not above the threshold value are not assigned to the comparable patients. In particular, the threshold value can be received as user input.

The inventors have recognized that, based on a comparison with a threshold value, the comparable patients can be determined based on an objective criterion. By receiving a threshold value, the user or interacting software can determine how close the conformity between the patients should be or how large the set of comparable patients should be compared to the set of second patients.

According to a further aspect of one or more embodiments of the invention, the method furthermore comprises determining a probability value for a side effect of medical treatment of the first patient based on the at least one comparable patient, in particular based on the side effects of similar medical treatments of the at least one comparable patient.

In this case, medical treatment is in particular medication, surgical intervention or other therapeutic and diagnostic methods that affect the respective patient.

The inventors have recognized that similar patients often respond in a similar way to medical treatment, i.e., in particular similar side effects of medical treatment can also occur. The use of the patient ontology enables the similarity to relate to a physiological similarity of the patients (height, age, weight, genomic data, transcriptomic data, proteomic data and/or metabolomic data), similarity with regard to an acute condition and/or similarity with regard to medical history. Therefore, side effects can be predicted with particular accuracy thereby supporting clinical decisions.

According to a further aspect of one or more embodiments of the invention, the method furthermore comprises determining a probability value for the outcome of medical treatment of the first patient based on the at least one comparable patient, in particular based on the outcome of similar medical treatments of the at least one comparable patient.

The inventors have recognized that similar patients often respond in a similar way to medical treatments, i.e., also in particular the outcome of medical treatment is comparable. The use of the patient ontology enables the similarity to relate to a physiological similarity of the patients (height, age, weight, genomic data, transcriptomic data, proteomic data and/or metabolomic data), similarity with regard to an acute condition and/or similarity with regard to medical history. Therefore, the probability of success of medical treatment can be predicted with particular accuracy thereby supporting medical decisions.

According to a further aspect of one or more embodiments of the invention, the patient ontology is based on genomic data from the first and/or the second patient data record, on epigenomic data from the first and/or the second patient data record, on transcriptomic data from the first and/or the second patient data record, on proteomic data from the first and/or the second patient data record and/or on metabolomic data from the first and/or the second patient data record. In particular, in this case, the first and/or the second patient ontology are based on genomic data from the first and/or the second patient data record, on epigenomic data from the first and/or the second patient data record, on transcriptomic data from the first and/or the second patient data record, on proteomic data from the first and/or the second patient data record and/or on metabolomic data from the first and/or the second patient data record. In particular, the first patient ontology can be based on said data. In particular, the second patient ontology can be based on said data. In particular, the common patient ontology can be based on said data. In particular, in this case, the medical ontology can be based on systems biology relationships.

Genomic data from a patient data record is in particular data from the patient data record relating to the genome of the respective patient. In this case, a genome in particular refers to the totality of the material carriers of the heritable information of a cell of an individual or also the totality of the heritable information (genes) of an individual. Genomic data can in particular comprise a base sequence ascertained by DNA sequencing. A base sequence in particular comprises a defined sequence of the nucleic bases adenine, guanine, thymine and cytosine.

Epigenomic data from a patient data record are in particular data from the patient data record relating to the epigenome of the respective patient. In this case, an epigenome describes the totality of epigenetic states and consists of a record of chemical changes to the DNA and histone proteins of an organism. Such changes can be passed down to an organism's offspring via transgenerational epigenetic inheritance. Changes to the epigenome can in particular result in changes to the structure of the chromatin and changes to the function of the genome. The epigenome is in particular involved in the regulation of gene expression, development, tissue differentiation and suppression of transposable elements. In contrast to the underlying genome, which remains largely static within an individual, the epigenome can in particular be dynamically alterable by environmental conditions.

Transcriptomic data from a patient data record is in particular data from the patient data record relating to the transcriptome of the respective patient. In this case, the transcriptome in particular refers to the genes transcribed in a cell at a specific time, i.e., the genes transcribed from DNA into RNA, i.e., the totality of all RNA molecules produced in a cell.

A transcriptome can in particular be determined based on the RT-PCR method (reverse transcriptase polymerase chain reaction) with degenerate primers, followed by DNA microarray or high-throughput DNA sequencing (RNA-seq or whole-transcriptome shotgun sequencing). An alternative option is serial analysis of gene expression (SAGE) and its further development SuperSAGE.

Proteomic data from a patient data record are in particular data from the patient data record relating to the proteome of the respective patient. In this case, the proteome refers in particular to the totality of all proteins in a living being, a tissue, a cell and/or a cell compartment, in particular under precisely defined conditions and/or at a specific time. In this case, the proteome can in particular be understood as a state of equilibrium between synthesis and degradation of proteins and is constantly subject to changes in composition. These changes are controlled in the course of spatiotemporal gene expression via complex regulatory processes and are significantly influenced by environmental stimuli, diseases, active substances and medication.

Various methods are known for separation (for example, serial extraction, serial precipitation, chromatography and/or electrophoresis) and for identification or characterization (for example, mass spectrometry, NMR spectroscopy, protein sequencing by Edman degradation, staining with antibodies or other selective ligands, direct or coupled enzymatic detection and/or phenotypic detection) of individual protein species in the proteome.

Metabolomic data from a patient data record are in particular data from the patient data record relating to the metabolome of the respective patient. In this case, the metabolome in particular refers to the totality of all characteristic metabolic properties of a cell, tissue or organism. The metabolome can in particular comprise flow rates (=turnover rates), metabolite levels and enzyme activities of individual metabolic pathways, interactions between different metabolic pathways and/or compartmentalization of different metabolic pathways within the cells.

The inventors have recognized that genomic data, epigenomic data, transcriptomic data, proteomic data and/or metabolomic data from the first patient data record allow a high level of information about the physiology and/or metabolism of a patient. Therefore, this data can in particular be used to ascertain effects of medical treatment very efficiently.

According to a further aspect of one or more embodiments of the invention, the medical ontology maps at least one of the following influences:
influence of a human genome on the human genome, epigenome, transcriptome, proteome and/or metabolome,
influence of a human epigenome on the human genome, epigenome, transcriptome, proteome and/or metabolome,
influence of a human transcriptome on the human genome, epigenome, transcriptome, proteome and/or metabolome,
influence of a human proteome on the human genome, epigenome, transcriptome, proteome and/or metabolome,
influence of a human metabolome on the human genome, epigenome, transcriptome, proteome and/or metabolome.

According to a further aspect of one or more embodiments of the invention, the first or the second patient ontology maps at least one of the following influences: influence of a patient's genome on the patient's transcriptome, influence of a patient's genome on the patient's proteome, influence of a patient's genome on the patient's metabolome, influence of a patient's transcriptome on the patient's proteome, influence of a patient's transcriptome on the patient's metabolome, and/or influence of a patient's proteome on the patient's metabolome.

The inventors have recognized that these causal relationships in particular enable conclusions to be drawn from known data about the patient of a first type regarding possibly missing data from a patient of a second type. For example, existing transcriptomic data from a patient and causal relationships between the patient's transcriptome and proteome can be used to draw conclusions regarding proteomic data from the patient. Alternatively, it is possible to acquire and take account of deviations of a patient from known causal relationships that may, for example, be caused by pathological changes or mutation. Both effects can result in the similarity measure being better able to take account of individual characteristics and therefore enable patients who are actually similar to be found.

According to a further aspect of one or more embodiments of the invention, the patient ontology (in particular the first patient ontology, the second patient ontology and/or the common patient ontology) is based on one of the following types of data or data records:
genome sequence, germline mutations in the genome sequence and/or somatic mutations in the genome sequence of the first patient and/or the second patient,
gene expression of the first patient and/or the second patient,
pre-existing conditions and/or comorbidities of the first patient and/or the second patient,
symptoms occurring in the first patient and/or the second patient,
lifestyle of the first patient and/or the second patient, in particular alcohol consumption, tobacco consumption and/or drug consumption of the first patient and/or the second patient,
physiological characteristics of the first patient and/or the second patient, in particular height, weight, age, gender and/or ethnicity of the first patient and/or the second patient.

Simultaneously or alternatively, the medical ontology is based on one of the following types of data or data records:
human gene expression,
transcription factor binding sites, enhancer sites and/or splice sites with respect to the human genome and/or transcriptome,
amino acid sequences and/or protein domains with respect to the human proteome,
spatial positional relationship of elements of the human genome and/or proteome,
clinical annotations with respect to elements of the human genome, epigenome, transcriptome, proteome and/or metabolome
biological interaction pathways of the human genome, epigenome, transcriptome, proteome and/or metabolome in particular gene regulatory networks, metabolic pathways and/or signal transduction pathways,
interaction between conditions and symptoms in humans,
interaction between pharmaceutical products, treatable conditions and side effects.

A mutation is in particular a spontaneously occurring permanent change to the genetic material or the genome sequence. Germline mutations are in particular mutations that are inherited by offspring via the germline, germline mutations in particular relate to oocytes or sperm and their precursors before and during oogenesis or spermatogenesis. Somatic mutations are in particular mutations relating to somatic cells. Somatic mutations in particular affect the organism in which they occur but are not inherited by the offspring of the organism.

Gene expression in particular refers to the relationship between the genetic information (or genotype) of an organism and its phenotype. In particular, therefore, the gene expression refers to effects on an organism caused by a specific element of the genome.

A transcription factor binding site is a DNA binding site of a transcription factor. A transcription factor is in particular a protein that is important for the initiation of RNA polymerase in transcription. In particular, therefore, transcription factors describe effects of elements of the proteome on the transcriptome. An enhancer site is a section of DNA with one or more transcription factor binding sites. The binding of the one or more transcription factors to an enhancer site influences the attachment of the transcription complex to the promotor and hence enhances the transcription activity of a gene. A splice site describes the site of splicing during the transition from pre-Mrna to mature Mrna, wherein in particular introns are extracted.

An amino acid sequence is in particular the sequence of the different amino acids in a peptide, in particular the polypeptide chain of a protein. A protein domain is in particular a region of a protein with a stable, mostly compact, convolutional structure that is functionally and structurally (quasi-)independent of adjacent sections.

A spatial positional relationship of elements of the genome is in particular defined by a one-dimensional (position or distance with respect to the genome strand) or a three-dimensional (position or distance with respect to the convoluted or compressed gene) positional relationship of different genes on the chromosomes and/or the patient's genome, in particular by their one-dimensional or three-dimensional distance. A spatial positional relationship of elements of the proteome can also describe the one-dimensional and/or three-dimensional position of elements of a protein. A local proximity of these elements can in particular indicate a common change or a common presence or absence of these elements.

Clinical annotation with respect to elements of the human genome, epigenome, transcriptome, proteome and/or metabolome is in particular a suspected or proven effect of the presence, absence or change to this element on the human organism, in particular on the human phenotype.

A gene regulatory network is in particular a collection of deoxyribonucleic acid segments in a cell that interact directly or indirectly with one another (through their ribonucleic acid and protein messengers) or with other substances in the cell, wherein they control the frequency, with which the genes in the network are transcribed into Mrna. A metabolic pathway in particular describes the assembly/degradation and conversion process in human cells. Metabolic pathways are in particular the defined sequence of biochemical reactions (in particular those catalyzed by enzymes). A signal transduction pathway in particular refers to a process via which human cells respond to (in particular external) stimuli, convert them, transmit them as a signal into the cell interior and lead to the cellular effect via a signal chain.

An interaction between a condition and a symptom in humans can in particular be a causal relationship between the condition and the symptom, in particular the information that a specific condition triggers one or more symptoms with a certain probability. For example, the condition influenza can trigger fever as a symptom. An interaction between a pharmaceutical product and a condition can describe the fact or the observation that this pharmaceutical product leads (at least with a certain probability) to an improvement or cure of this condition. An interaction between a pharmaceutical product and a side effect can describe the fact or the observation that this pharmaceutical product triggers (at least with a certain probability) this side effect.

The inventors have recognized that said data and information can be represented in a medical ontology or a patient ontology and thus represent complex causal relationships in a systematic manner. Based on the systematization in a medical ontology, it is then possible to determine a similarity measure efficiently, while at the same time taking account of complex causal relationships.

In a further aspect, one or more embodiments of the invention relates to a determination system for determining a similarity measure, wherein the similarity measure describes a similarity between a first patient and a second patient, comprising an interface and a computing unit,
wherein the interface is embodied to receive a first patient data record, wherein the first patient data record is assigned to the first patient;
wherein the interface is embodied to receive a second patient data record, wherein the second patient data record is assigned to the second patient;
wherein the interface or the computing unit are embodied to receive or determine a medical ontology, wherein the medical ontology is independent of the first patient data record and the second patient data record;
wherein the computing unit is embodied to determine a patient ontology based on the medical ontology and furthermore based on the first patient data record and/or the second patient data record;
wherein the computing unit is embodied to determine the similarity measure based on the patient ontology.

Such a determination system can in particular be embodied to execute the above-described methods according to one or more embodiments of the invention for determining a similarity measure and the aspects thereof. The determination system is embodied to execute these methods and the aspects thereof in that the interface and the computing unit are embodied to execute the corresponding method steps.

In a further aspect, one or more embodiments of the invention relates to a computer program product with a computer program, which can be loaded directly into a memory of a determination system, with program sections for executing all steps of the method for determining a similarity measure and the aspects thereof when the program sections are executed by the determination system.

In a further aspect, one or more embodiments of the invention relates to a computer-readable storage medium on which program sections that are readable and executable by a determination system are stored in order to execute all steps of the method for determining a similarity measure and the aspects thereof when the program sections are executed by the determination system.

A largely software-based implementation has the advantage that determination systems that have already been used can be retrofitted in a simple manner by a software update in order to operate in the manner according to one or more embodiments of the invention. In addition to the computer program, such a computer program product can comprise additional parts, such as, for example, documentation and/or additional components and hardware components, such as, for example, hardware keys (dongles etc.) for using the software.

### BRIEF DESCRIPTION OF THE DRAWINGS

The above-described characteristics, features and advantages of this invention and the manner in which they are achieved will become clearer and more plainly comprehensible in conjunction with the following description of the exemplary embodiments explained in more detail in conjunction with the drawings. This description does not restrict the invention to these exemplary embodiments. The same components are provided with identical reference symbols in different figures. The figures are generally not to scale. The figures show:

FIG. 1 illustrates a first possible connection between a medical ontology, a common patient ontology, and a first and second patient ontology,

FIG. 2 illustrates a second possible connection between a medical ontology and a first and second patient ontology,

FIG. 3 illustrates a first exemplary embodiment of a medical ontology, a common patient ontology and a first and second patient ontology in the field of systems biology,

FIG. 4 illustrates a second exemplary embodiment of a medical ontology, a common patient ontology and a first and second patient ontology in the field of classification systems for diagnoses,

FIG. 5 illustrates a first exemplary embodiment of a method for determining a similarity measure,

FIG. 6 illustrates a second exemplary embodiment of a method for determining a similarity measure,

FIG. 7 illustrates a second exemplary embodiment of a method for determining a similarity measure,

FIG. 8 illustrates a first possible extension of the method for determining a similarity measure according to the first exemplary embodiment, the second exemplary embodiment and/or the third exemplary embodiment,

FIG. 9 illustrates a second possible extension of the method for determining a similarity measure according to the first exemplary embodiment, the second exemplary embodiment and/or the third exemplary embodiment, and

FIG. 10 illustrates a determination system.

### DETAILED DESCRIPTION

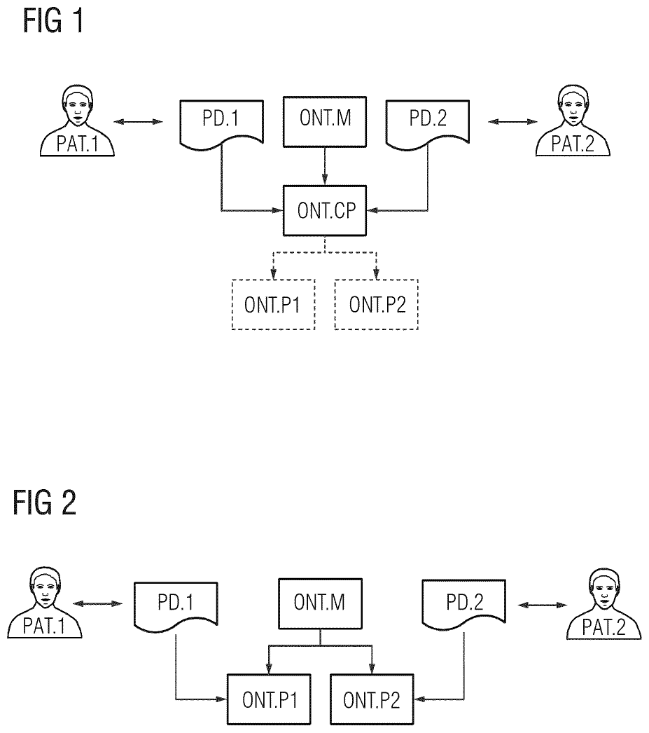

FIG. 1 and FIG. 2 show possible relationships between a medical ontology ONT.M, a common patient ontology ONT.CP, a first and second patient ontology ONT.P1, ONT.P2 and a first and second patient data record PD.1, PD.2. In this case, the first patient data record PD.1 relates to a first patient PAT.1 and the second patient data record PD.2 relates to a second patient PAT.2. In this case, the first patient PAT.1 and the second patient PAT.2 are different.

FIG. 1 is a schematic depiction of the creation of a common patient ontology ONT.CP. In this exemplary embodiment, the common patient ontology ONT.CP is based on both a medical ontology ONT.M and the first patient data record PD.1 and the second patient data record PD.2. In addition, the common patient ontology ONT.CP can also be based on patient data records (not depicted here).

In this exemplary embodiment, the first patient ontology ONT.P1 is derived from the common patient ontology ONT.CP. Herein, the first patient ontology ONT.P1 comprises concepts of medical ontology ONT.M as well as relevant data from the first patient PAT.1 (based on the first patient data record PD.1). Furthermore, the second patient ontology ONT.P2 is derived from the common patient ontology ONT.CP. For this purpose, the second patient ontology ONT.P2 comprises concepts of medical ontology ONT.M as well as relevant data from the second patient PAT.2 (based on the second patient data record PD.2).

In this case, the use of the first patient ontology ONT.P1 and the second patient ontology ONT.P2 is optional for the determination of the similarity measure. Since all relevant data is already present in the common patient ontology ONT.CP, the common patient ontology ONT.CP can be equally used to determine the similarity measure.

FIG. 2 is a schematic depiction of the creation of a first patient ontology ONT.P1 and a second patient ontology ONT-O2 without using a common patient ontology ONT.CP. In this exemplary embodiment, the first patient ontology ONT.P1 is derived from the medical ontology ONT.M using the first patient data PD.1. Furthermore, the second patient ontology ONT.P2 is derived from the medical ontology ONT.M using the second patient data PD.2.

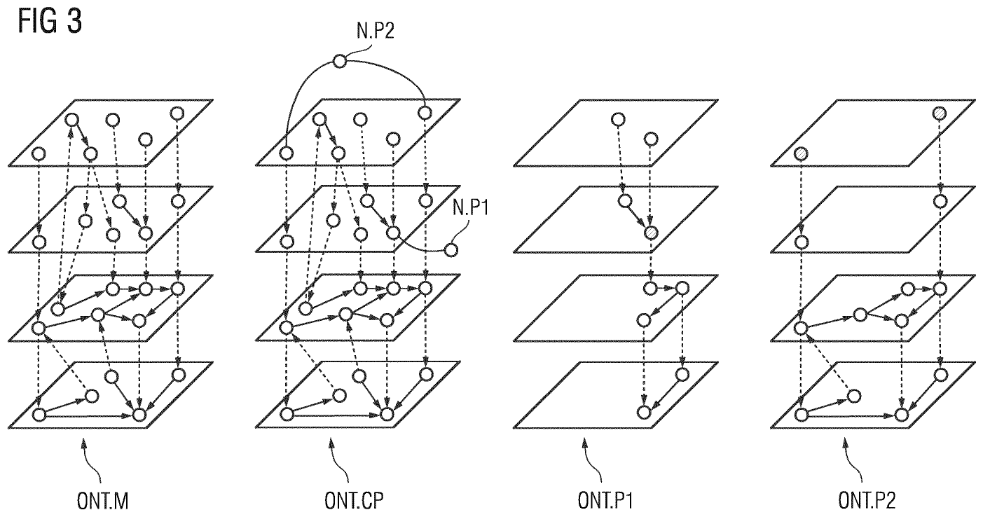

FIG. 3 depicts a first exemplary embodiment of a medical ontology ONT.M, a common patient ontology ONT.CP and a first and second patient ontology ONT.P1, ONT.P2 in the field of systems biology.

In this case, the medical ontology ONT.M links and structures knowledge about a human genome, transcriptome, proteome and metabolome. In this case, this knowledge is depicted in the form of a directed graph. In this case, a node in each case represents an element of the genome, transcriptome, proteome or metabolome. For a better graphical depiction, the nodes are depicted in four levels according to these four elements (from top to bottom: genome, transcriptome, proteome, metabolome), however, this arrangement is irrelevant for the performance of the method. The edges of the directed graph represent influences or interactions between these elements. These influences or interactions can be present between elements at the same level (for example, proteome to proteome), as well as between elements at different levels (for example, genome to transcriptome, proteome to genome). The present graph can be interpreted as a node-colored graph, i.e., each node is assigned an attribute that makes the node distinguishable from other nodes (for example, a specific protein at a node denoting an element of the proteome, or a specific gene/specific gene mutation at a node denoting an element of the genome).

In this exemplary embodiment, the common patient ontology ONT.CP is constructed by introducing a patient node N.P1, N.P2 for each patient PAT.1, PAT.2 or for each patient data record PD.1, PD.2. In this case, the patient nodes N.P1, N.P2 are not connected to one another by an edge, but only to nodes that were already present in the medical ontology ONT.M graph. Here, a connection between a patient node N.P1, N.P2 and a node corresponds to the information that the element corresponding to the connected node is contained or referenced in the respective patient data record PD.1, PD.2. For example, in the exemplary embodiment depicted, the first patient node N.P1 is connected to exactly one node corresponding to an element of the transcriptome, this is equivalent to the fact that the first patient data record N.P1 contains information that the first patient PAT.1 has this element of the transcriptome. Furthermore, in the exemplary embodiment depicted, the second patient node N.P2 is connected to two nodes corresponding to elements of the genome; this is equivalent to the fact that the second patient data record N.P2 contains information that the second patient PAT.2 has the corresponding two genes and/or gene mutations.

FIG. 3 furthermore depicts a first patient ontology ONT.P1 and a second patient ontology ONT.P2. In the exemplary embodiment depicted, the patient ontologies ONT.P1, ONT.P1 depicted are in each case defined by a subgraph of the medical ontology ONT.M, but, alternatively, the complete medical ontology ONT.M supplemented by the patient node N.P1, N.P2 could also be used as the patient ontology ONT.P1, ONT.P2.

In this case, the patient ontology ONT.P1, ONT.P2 can be determined based on the common patient ontology ONT.CP or also directly based on the medical ontology ONT.M and the respective patient data record PD.1, PD.2.

In the present exemplary embodiment, the patient ontology ONT.P1, ONT.P2 is constructed in such a way that the patient ontology ONT.P1, ONT.P2 comprises all nodes connected to the patient node N.P1, N.P2 (referred to as base nodes). Furthermore, the patient ontology ONT.P1, ONT.P2 comprises all nodes that can be reached from the base nodes when the directed edges are followed according to their direction (these nodes correspond to the elements that are conditioned or favored by the elements conditioned or favored by the base nodes). Furthermore, the patient ontology ONT.P1, ONT.P2 comprises all nodes that can be reached by the base nodes when the directed edges are followed opposite to their direction (these nodes correspond to the elements that condition or favor the elements corresponding to the base node).

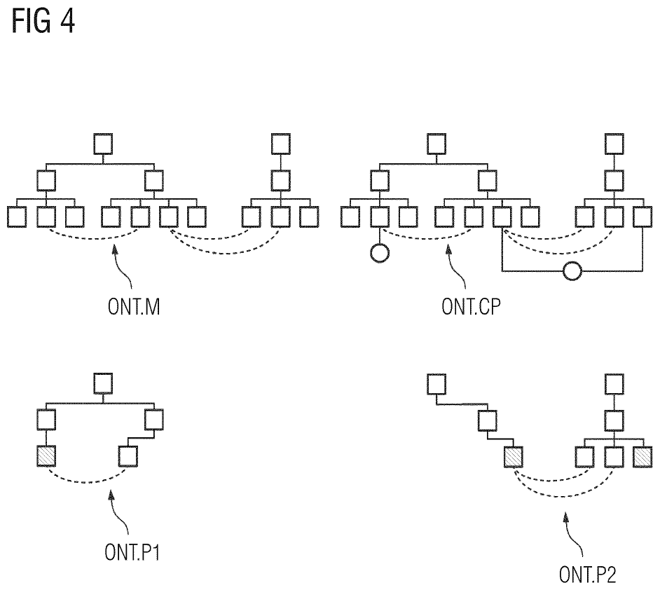

FIG. 4 depicts a second exemplary embodiment of a medical ontology ONT.M, a common patient ontology ONT.CP and a first and second patient ontology ONT.P1, ONT.P2 in the field of classifications for medical diagnoses. In this exemplary embodiment, the classification corresponds schematically to the ICD-10 classification, even though it is only shown in a simplified and schematic form here.

In this case, the medical ontology ONT.M links and structures knowledge about possible diagnoses and their interactions in humans. In this case, an ICD-10 code has a hierarchical structure. For example, the ICD-10 code Q90.0 (Trisomy 21, meiotic nondisjunction) is sorted into the group Q90 (Down syndrome), which is in turn sorted into the group Q90-99 (Chromosomal anomalies, not elsewhere classified), which is in turn sorted into chapter XVII/Q00-99 (Congenital malformations, deformities and chromosomal anomalies). The hierarchical arrangement is represented by the tree structure in FIG. 4.

Furthermore, in the medical ontology ONT.M, elements (also on different levels) can be linked to one another by cross-references. In particular, specific diseases can be mapped by dual classifications. In this case, a primary classification can be made according to the etiology and a secondary classification according to the organ manifestation. In the ICD-10 system, the primary key can be marked with a cross (+) and the secondary key can be marked with an asterisk. For example, tuberculous meningitis has the ICD-10-code A17.0+ (Etiology: infectious disease) and G01* (Organ manifestation: disease of the nervous system). There is, for example, a similar connection between the ICD-10 codes E10.30+ (Diabetes mellitus type I with ophthalmic complication, not referred to as uncontrolled) and H36.0* (Retinopathia diabetica). These cross-references are indicated by dashed arrows in FIG. 4.

In this exemplary embodiment, the common patient ontology ONT.CP is constructed by introducing a patient node N.P1, N.P2 for each patient PAT.1, PAT.2 or for each patient data record PD.1, PD.2. In this case, the patient nodes N.P1, N.P2 are not connected to one another by an edge, but only to nodes that were already present in the graph of the medical ontology ONT.M. Here, a connection between a patient node N.P1, N.P2 and a node corresponds to the information that a diagnosis with the ICD-10 code of the node has been made with respect to the patient PAT.1, PAT.2 and is contained or referenced in the patient data record PD.1, PD.2. For example, in the exemplary embodiment depicted, the first patient node N.P1 is connected to exactly one node corresponding to an ICD-10 code, this is equivalent to the first patient data record N.P1 containing information that the first patient PAT.1 has been diagnosed based on this ICD-10 code. Furthermore, in the exemplary embodiment depicted, the second patient node N.P2 depicted with two nodes corresponding to ICD-10 codes, this is equivalent to the second patient PAT.1 having been diagnosed based on these two ICD-10 codes.

FIG. 4 furthermore depicts a first patient ontology ONT.P1 and a second patient ontology ONT.P2. In the exemplary embodiment depicted, the patient ontologies ONT.P1, ONT.P1 are in each case defined by a subgraph of the medical ontology ONT.M, alternatively, it would also be possible to use the complete medical ontology ONT.M supplemented by the patient node N.P1, N.P2 as the patient ontology ONT.P1, ONT.P2. The patient ontologies ONT.P1, ONT.P2 can be created and/or determined analogously to the methods depicted and described with respect to FIG. 3.

FIG. 5 depicts a first exemplary embodiment of a method for determining a similarity measure, wherein the similarity measure describes a similarity between a first patient and a second patient.

The first steps of the method are receiving REC-PD.1 a first patient data record PD.1, wherein the first patient data record PD.1 is assigned to the first patient PAT.1, and receiving REC-PD.2 a second patient data record PD.2, wherein the second patient data record PD.2 is assigned to the second patient PAT.2. Furthermore, in this exemplary embodiment, a medical ontology ONT.M is received or determined REC-DET-ONT.M, wherein the medical ontology ONT.M is independent of the first patient data record PD.1 and the second patient data record PD.2. In this first exemplary embodiment, the medical ontology ONT.M is received. In this case, all of the aforementioned steps are executed via an interface IF of a determination system SYS. In this case, the order of these three steps is irrelevant; they can be performed in pairs in any order or even simultaneously.

In the first exemplary embodiment, in a first variant, the medical ontology ONT.M is an ontology in the field of systems biology, as depicted in FIG. 3. In this case, the patient data records PD.1, PD.2 correspond to database entries from an EMR (electronic medical record) from an electronic health record and/or a hospital's IT systems, for example, an LIS (laboratory information system), a HIS (hospital information system), an RIS (radiology information system) and/or a PACS (picture archiving and communication system). In particular, the patient data records PD.1, PD.2 contain systems biology information about the patient, for example, genomic mutations, abnormalities in the transcriptome, detected proteins or metabolites).

In a second variant, the medical ontology ONT.M is an ontology for the classification of medical diagnoses, as depicted in FIG. 4. In this case, the patient data records PD.1, PD.2 in particular correspond to the database entries described with respect to the first variant. In particular, the patient data records PD.1, PD.2 comprise diagnoses relating to the patient PAT.1, PAT.2 that can be classified via the classification schema.

In a third variant, the medical ontology ONT.M is a combination of an ontology in the field of systems biology, as depicted in FIG. 3, and an ontology for the classification of medical diagnoses, as depicted in FIG. 4. In this case a combination can take place in that the two ontologies are used side by side without interaction or connections; but advantageously interactions between systems biology information and associated diagnoses are also acquired. In this case, the patient data records PD.1, PD.2 in particular correspond to the database entries described with respect to the first variant, which additionally comprise diagnoses relating to the patient PAT.1, PAT.2 that can be classified via the classification schema. In further variants, it is, of course, also possible to use other medical ontologies ONT.M or combinations thereof.

A further step of the method depicted is determining DET-ONT.CP a common patient ontology ONT.CP based on the medical ontology ONT.M, the first patient data record PD.1 and the second patient data record PD.2.

In this exemplary embodiment, both the medical ontology ONT.M and the common patient ontology ONT.CP comprise a graph, in this case, the graph of the medical ontology ONT.M is a subgraph of the common patient ontology ONT.CP. In addition to the subgraph, the medical ontology comprises a node N.P1, N.P2 for each type of patient data PD.1, PD.2.

In this case, when determining DET-ONT.CP the common patient ontology ONT.CP, this node N.P1, N.P2 is connected, based on the patient data PD.1, PD.2, to the nodes of the subgraph that represent elements of the medical ontology ONT.M that are relevant for the patient PAT.1, PAT.2 or can be found in the patient data PD.1, PD.2.

In the first variant, for example, genomic mutations, abnormalities in the transcriptome, detected proteins or detected metabolites relating to the patient PAT.1, PAT.2 are extracted and the additional nodes N.P1, N.P2 are connected to the corresponding elements. In the second variant, the additional nodes N.P1, N.P2 are in each case connected to nodes of the classification ontology that represent the diagnoses for the respective patient PAT.1, PAT.2. In the third variant, the additional nodes N.P1, N.P2 are connected to the corresponding nodes of the systems biology ontology and the classification ontology.

In the first exemplary embodiment depicted, further the similarity measure is determined DET-SV based on the common patient ontology ONT.CP. In this case, the similarity measure is based on the probability of an edge (or an edge probability) between the first node N.P1 corresponding to the first patient data PD.1 or to the first patient PAT.1, and the second node N.P2 corresponding to the second patient data PD.2 or to the second patient PAT.2. In this case, the similarity measure is in particular identical to the probability of the edge.

In this case, the probability of the edge can be determined using different methods (also called link prediction):

A first method is the use of the number of common neighbors of the first and the second node N.P1, N.P2. Let v1 denote the first node N.P1, v2 the second node N.P2, and NN(v1) the set of nodes in the graph which are connected to v1 via an edge. Then, NN(v1)∩NN(v2) is the set of nodes in the graph that are connected to both v1 and v2 via an edge and the probability p(v1, v2) of the edge is then defined up to a normalization factor by p(v1, v2)˜|NN (v1)∩NN(v2)1. Without a normalization factor, |NN(v1)∩NN(v2)| could also be used directly as a similarity measure.

A further method is the use of the Jaccard measure which relates the number of common neighbors to the total number of neighbors. In this case, the probability is defined by p(v1, v2)=|NN(v1)∩NN(v2)|/|NN(v1)UNN(v2)1, wherein ∩ denotes the intersection and U the union.

A further method is the use of the Adamic-Adar measure; in this case, the probability is defined by p(v1, v2)=Σw∈NN(v1)∩NN(v2) [log NN(w)]−1. This measure in particular takes account of the fact that connecting nodes with a large neighborhood of nodes should contribute less to the probability than nodes with only a few connections. A further possibility is the use of a probability based on the Katz measure.

The probability of an edge or the similarity measure can also be based on vertex embedding and/or graph embedding of the common patient ontology ONT.CP. In this case, the graph of the common patient ontology ONT.CP can in particular be embedded into a two-dimensional or a higher-dimensional space. The probability of an edge can then in particular be based on the (Euclidean) distance between the first node N.P1 and the second node N.P2 in the embedding space. For example, the probability can be the ratio of this distance and the maximum distance between any two edges in the embedding space.

A final optional step of the first exemplary embodiment depicted is the provision PROV-SM of the similarity measure. In this case, the provision can in particular comprise displaying, transmitting and/or storing the similarity measure. The similarity measure can in particular also be used to support a medical decision; in particular, it enables decisions about a medical diagnosis and/or therapy for a patient to be compared with similar cases.

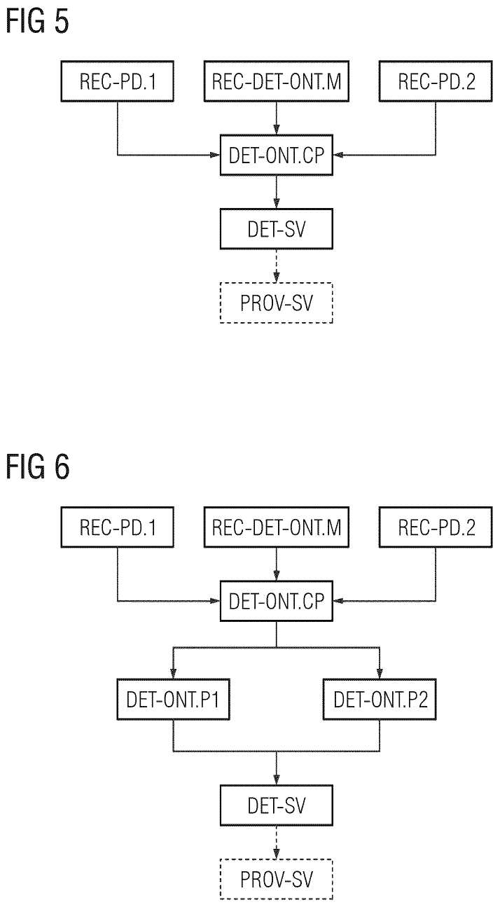

FIG. 6 depicts a second exemplary embodiment of a method for determining a similarity measure, wherein the similarity measure is a similarity between a first patient PAT.1 and a second patient PAT.2.

The steps of receiving REC-PD.1, REC-PD.2 the first and the second patient data record REC-PD.1 and receiving or determining REC-DET-ONT.M the medical ontology ONT.M are identical to the first exemplary embodiment, and can in particular include all advantageous embodiments and developments. In particular, the medical ontology ONT.M can include the variants described with respect to the first exemplary embodiment.

The determination DET-ONT.CP of the common patient ontology ONT.CP is based on the medical ontology ONT.M, the first patient data record PD.1 and the second patient data record PD.2 is also identical to the first exemplary embodiment.

In contrast to the first exemplary embodiment, in the second exemplary embodiment shown, the determination DET-ONT.P1 of a first patient ontology ONT.P1 and the determination DET-ONT.P2 of a second patient ontology ONT.P2 in each case takes place based on the common patient ontology ONT.CP. In this exemplary embodiment, this determination DET-ONT.P1 takes place in each case according to the manner depicted in FIG. 3 and FIG. 4.

In this second exemplary embodiment, the determination DET-SV of the similarity measure takes place based on the first patient ontology ONT.P1 and the second patient ontology ONT.P2. In particular, the determination DET-SV can take place based on a comparison of the first patient ontology ONT.P1 and the second patient ontology ONT.P2. In this case, the first patient ontology ONT.P1 comprises a first graph and the second patient ontology ONT.P2 comprises a second graph. In this case, the first graph can in particular comprise a subgraph of the medical ontology ONT.M and, in this case, the second graph can also comprise a further subgraph of the medical ontology ONT.M (in this case, the first graph and the second graph can in each case additionally comprise a patient-specific node N.P1, N.P2). In particular, in this case, the first graph can be identical to a subgraph with respect to the medical ontology ONT.M and the second graph can be identical to a further subgraph with respect to the medical ontology ONT.M. In this case, the similarity measure is then based on a similarity between the first graph and the second graph.

One possible method is to use the graph edit distance of the first graph and the second graph as a similarity measure. Let g1 denote the first graph, g2 the second graph, and P(g1, g2) the set of edit paths which transform the first graph g1 into the second graph, then the graph edit distance can be calculated as:

$$GED ( {g}_{1} , {g}_{2} ) = {min}_{( {e}_{1} , … {,}_{} {e}_{k} ) ∈ P ( {g}_{1} , {g}_{2} )} {\sum }_{i = 1}^{k} c ( {e}_{i} )$$

here, (e1, . . . , ek) is an edit path comprising the elementary steps e1 to ek, and c(ei) denotes the weight of the i-th elementary step, which can in particular be 1 for each elementary step so that the value corresponds to the sum of the number of elementary steps of the edit path. Elementary steps are the insertion of a node, the removal of a node, a change to the label or color of a node, the insertion of an edge and/or the deletion of an edge.

A further possibility is the use of the maximum common subgraph distance of the first graph and the second graph as a similarity measure. Here, a “maximum common subgraph” is the subgraph of the first and a second graph with the largest number of nodes. The similarity measure or the maximum common subgraph distance can be defined by the ratio of this maximum number of nodes and the number of nodes in the first and/or in the second graph.

A further possibility consists in the fact that the similarity measure is based on vertex embedding and/or graph embedding of the first graph and the second graph. In this case, the similarity measure can in particular be based on a distance between the first graph and the second graph in the embedding vector space and in particular can be indirectly proportional to this distance.

A further possibility is the use of a trained function to determine the similarity measure. In this case, this trained function can in each case work on embedded or non-embedded graphs. For example, a neural network can be used to factorize the adjacency matrix of a graph (such as, for example, described in the document: G. Dziugaite, D. Roy, Neural Network Matrix Factorization, arXiv:1511.06443 (2015)), in order to determine the similarity of two graphs (such as, for example, described in the document: K. Duang et al., Symmetric Nonnegative Matrix Factorization for Graph Clustering, doi:10.1137/1.9781611972825.10 (2012)). Furthermore, it is, for example, possible to use (relational) graph neural networks (such as, for example, those described in the document: M. Schlichtkrull et al., Modeling Relational Data with Graph Convolutional Networks, arXiv:1703.06103 (2017)).

A further possibility is to use trained functions in which two graphs (for example, an adjacency matrix and/or an embedding of the respective graphs) are used as input data, and which provide a similarity measure as output data. For example, it is possible to apply the method described in the document: Y. Bai et al., SimGNN: A Neural Network Approach to Fast Graph Similarity Computation WSDM'19 (2019), https://doi.org/10.1145/3289600.3290967 to determine the graph edit distance, which can then serve as the basis for a similarity measure.

A final optional step of the second exemplary embodiment depicted is the provision PROV-SM of the similarity measure. In this case, the provision can in particular comprise displaying, transmitting and/or storing the similarity measure. The similarity measure can in particular also be used to support a medical decision; in particular, it enables decisions about a medical diagnosis and/or therapy for a patient to be compared with similar cases.

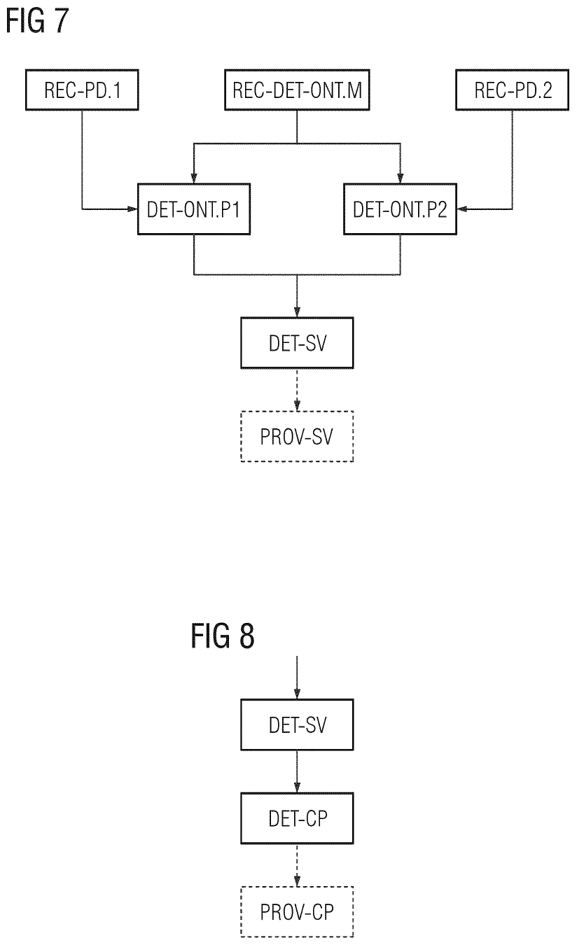

FIG. 7 depicts a third exemplary embodiment of a method for determining a similarity measure, wherein the similarity measure describes a similarity between a first patient PAT.1 and a second patient PAT.2.

The steps of receiving REC-PD.1, REC-PD.2 the first and the second patient data record REC-PD.1 and receiving or determining REC-DET-ONT.M the medical ontology ONT.M are identical to the first exemplary embodiment and can in particular include all advantageous embodiments and developments. In particular, the medical ontology ONT.M can include the variants described with respect to the first exemplary embodiment.

In the third exemplary embodiment, in a first variant, the medical ontology ONT.M is an ontology in the field of systems biology, as depicted in FIG. 3 or as explained in the first variant of the first exemplary embodiment. In a second variant of the third exemplary embodiment, the medical ontology ONT.M is an ontology for the classification of medical diagnoses, as depicted in FIG. 4 or as explained in the second variant of the first exemplary embodiment. In a third variant of the third exemplary embodiment, the medical ontology ONT.M is a combination of an ontology in the field of systems biology, as depicted in FIG. 3, and an ontology for the classification of medical diagnoses, as depicted in FIG. 4, or as described in the third variant of the first exemplary embodiment. Furthermore, it is, of course, also possible to use other medical ontologies ONT.M or combinations thereof. In this case, the structure of the patient data records PD.1, PD.2 in the third exemplary embodiment corresponds to the structure of the patient data records PD.1, PD.2 in the respective variants of the first exemplary embodiment.

In contrast to the first and second exemplary embodiments, no common patient ontology ONT.CP is determined in the third exemplary embodiment. Instead, the third exemplary embodiment comprises determining DET-ONT.P1 a first patient ontology ONT.P1 based on the medical ontology ONT.M and the first patient data record PD.1 and determining DET-ONT.P2 a second patient ontology ONT.P2 based on the medical ontology ONT.M and the second patient data record PD.2. In this case, the first patient ontology ONT.P1 is not based on the second patient data record PD.2, and the second patient ontology ONT.P2 is not based on the first patient data record PD.1.

In this exemplary embodiment, both the medical ontology ONT.M and the first and the second patient ontology ONT.P1, ONT.P2 comprise a graph. In this case, the first graph can in particular comprise a subgraph of the medical ontology ONT.M; in this case, the second graph can also comprise a further subgraph of the medical ontology ONT.M (in this case, the first graph and the second graph can in each case additionally comprise a patient-specific node N.P1, N.P2). In particular, in this case, the graph of the first patient ontology ONT.P1 is in particular identical to a subgraph of the graph of the medical ontology ONT.M and the graph of the second patient ontology ONT.P2 is in particular identical to a further subgraph of the graph of the medical ontology ONT.M. To determine DET-ONT.P1, DET-ONT.P2 the patient ontologies ONT.P1, ONT.P2, in this case, in particular those nodes in the graph of the patient ontologies ONT.P1, ONT.P2, are marked that represent elements of the medical ontology ONT.M that are relevant for the patient PAT.1, PAT.2 or can be found in the patient data PD.1, PD.2.

In the first variant, for example, genomic mutations, abnormalities in the transcriptome, detected proteins or detected metabolites relating to the patient PAT.1, PAT.2 are extracted and the corresponding nodes are marked. In the second variant, in particular nodes of the classification ontology are connected that represent diagnoses for the respective patient PAT.1, PAT.2. In the third variant, the method of the first two variants is combined.

Based on the marked nodes, it is then possible to determine the subgraph corresponding to the patient ontology ONT.P1, ONT.P2 by marking or selecting further nodes according to established rules, which, together with the edges between these nodes, form the subgraph. For example, the nodes in the first or a n-th neighborhood of the originally marked node can be added to the subgraph. In particular, in the case of directed graphs, for this purpose, edges can also be traversed in only one direction or against the established direction. Further examples of the generation of the subgraph corresponding to the patient ontologies ONT.P1, ONT.P2 are described with respect to FIG. 3 and FIG. 4.

In this third exemplary embodiment, the similarity measure is determined based on the first patient ontology ONT.P2 and the second patient ontology ONT.P2 analogously to the procedure described in the second exemplary embodiment.

A final optional step of the first exemplary embodiment depicted is the provision PROV-SM of the similarity measure. In this case, the provision can in particular comprise displaying, transmitting and/or storing the similarity measure. The similarity measure can in particular also be used to support a medical decision; in particular, it enables decisions about a medical diagnosis and/or therapy for a patient to be compared with similar cases.

FIG. 8 depicts a first possible extension of the method for determining a similarity measure according to the first exemplary embodiment, the second exemplary embodiment and/or the third exemplary embodiment.

In the first extension depicted, a similarity measure is determined DET-SV for a plurality of second patient data records PD.2, wherein the second patient data records PD.2 are assigned to a plurality of second patients PAT.2. If a common patient ontology ONT.CP is determined in advance, this can in particular be based on the plurality of second patient data records PD.2, in particular in that the corresponding graph of the common patient ontology ONT.CP comprises a second node N.P2 for each of the second patient data records. Furthermore, if a second patient ontology ONT.P2 is determined in advance, in this case, a plurality of second patients ontologies ONT.P2 can be determined, wherein each of the second patient ontologies ONT.P2 corresponds to one of the second patient data records. In particular, therefore, a similarity measure is then assigned to each second patient of the plurality of second patients.

The first extension depicted furthermore comprises determining DET-CP a set of comparable patients based on the determined similarity measures, wherein the set of comparable patients is a subset of the plurality of second patients PAT.2, and wherein in particular each of the comparable patients is similar to the first patient PAT.1.

If the similarity measures determined can be sorted (i.e., “smaller”, “equal to” and “greater than” is meaningfully defined with respect to similarity measures), it is in particular possible to select as comparable patients the second patients PAT.2 whose association similarity measure is above a predefine threshold value. If, for example, the similarity measure is a real number between 0 and 1, wherein 1 corresponds to a maximum similarity, such a threshold value can, for example, be defined as 0.9. Alternatively, it is also possible for a predefined number of second patients PAT.2 to be selected as comparable patients, in particular the second patients PAT.2 with the greatest associated similarity measures.

Comparisons with threshold values are also possible if the similarity measures cannot be sorted. For example, a similarity measure can be described by a tuple or a vector of numbers, wherein each entry can cover a different aspect of similarity or has been calculated in a different way. In this case, a threshold value can also be defined by a tuple or a vector with the same number of elements. A similarity measure can then be above the threshold value if all components of the similarity measure are above the respective components of the threshold value or if a predefined number of the components of the similarity measure are above the respective components of the threshold value. Alternatively, it is also possible for a norm of the tuple or the vector to be compared with a scalar threshold value; herein, individual components can also be weighted differently in the calculation of the norm.

A final optional step of the first extension depicted is the provision PROV-CP of the set of comparable patients. In this case, the provision can in particular comprise displaying, transmitting and/or storing the set of comparable patients. The set of comparable patients can in particular also be used to support a medical decision, in particular, it enables decisions about a medical diagnosis and/or therapy for a patient to be compared with similar cases.

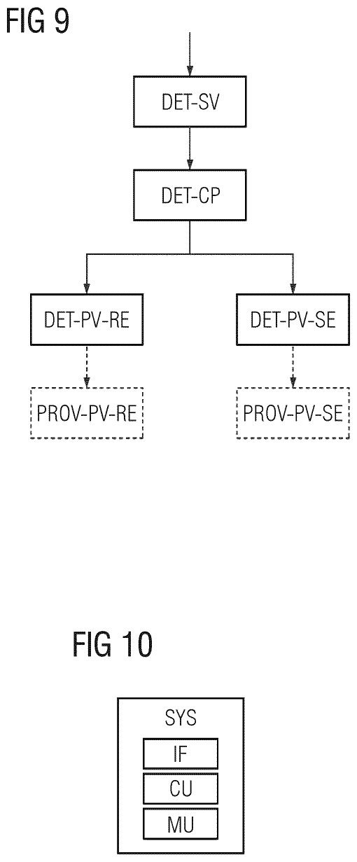

FIG. 9 depicts a second possible extension of the method for determining a similarity measure according to the first exemplary embodiment, the second exemplary embodiment and/or the third exemplary embodiment.

In the second possible extension, a set of comparable patients is also determined DET-CP based on similarity measures, wherein the set of comparable patients is a subset of the plurality of second patients PAT.2. This step can in particular be performed as with respect to the first extension depicted in FIG. 8.

The second extension furthermore comprises determining DET-PV-SE a probability value for a side effect of medical treatment of the first patient PAT.1 based on the set of comparable patients, in particular based on the side effects of similar medical treatments of the set of comparable patients. An optional step of the second extension depicted is then a provision PROV-PV-SE of the probability value for the side effect, wherein the provision PROV-SV-SE can comprise storing, transmitting and/or displaying this probability value. In particular, this probability value can be presented to the user in connection with the side effect and/or the treatment.

The second extension furthermore comprises determining DET-PV-RE a probability value for the outcome of medical treatment of the first patient PAT.1 based on the set of comparable patients, in particular based on the outcome of similar medical treatments of the set of comparable patients. An optional step of the second extension depicted is then the provision PROV-PV-RE of the probability value for the outcome, wherein the provision PROV-SV-RE can comprise storing, transmitting and/or depicting this probability value. In particular, this probability value can be depicted to a user in connection with the treatment.

In this case, the determination DET-PV-SE of the probability value for the side effect of the medical treatment and the determination DET-PV-RE of the probability value for the outcome of the medical treatment can be performed independently of one another. In particular, the order of these two steps is irrelevant; the two steps can also be performed at the same time. In particular, it is also possible for only one of these two steps to be performed.

For the determination DET-PV-SE, DET-PV-RE of both probability values, first, a first subset of patients in whom the medical treatment on the first patient PAT.1 has already been performed can be extracted from the set of comparable patients. This can in particular take place based on the patient data records PD.2 and the patient ontologies ONT.P2. This set serves as the population and its power can be denoted as N.

For the determination DET-PV-SE of the probability value for the side effect of the medical treatment, first, a second subset of patients in whom a side effect or a specific side effect has occurred with this medical treatment can then be extracted from this first subset of patients. This can in particular take place based on the patient data records PD.2 and the patient ontologies ONT.P2. The power of this second subset can be denoted as NSE. The probability value for the side effect can then in particular be denoted as NSE/N.

For the determination DET-PV-RE of the probability value for the outcome of the medical treatment, first, a third subset of patients in which this medical treatment has led to an outcome, i.e., in particular has cured or alleviated an underlying condition, can then be extracted from the first subset of patients. This can in particular take place based on the patient data records PD.2 and the patient ontologies ONT.P2. The power of this third subset can be denoted as NRE. The probability value for the side effect can then in particular be calculated as NRE/N.

FIG. 10 depicts a determination system SYS for determining a similarity measure. The determination system SYS depicted is embodied to execute a method according to one or more embodiments of the invention for determining a similarity measure. The determination system SYS comprises an interface IF, a computing unit CU and a memory MU.

The determination system SYS can in particular be a computer, a microcontroller or an integrated circuit. Alternatively, the determination system SYS can be a real or virtual network of computers (a technical term for a real network is cluster, a technical term for a virtual network is cloud). The determination system SYS can also be embodied as a virtual system that is executed on a real computer or a real or virtual network of computers (a technical term is virtualization).

An interface IF can be a hardware or software interface (for example, PCI-Bus, USB or Firewire). A computing unit CU can include hardware elements or software elements, for example, a microprocessor or an FPGA (field programmable gate array). A memory unit MU can be implemented as a non-permanent random-access memory (RAM) or as permanent mass storage (hard disk, USB stick, SD card, solid state disk).

The interface IF can in particular comprise a plurality of sub-interfaces that execute different steps of the respective methods. In other words, the interface IF can also be understood as being a large number of interfaces IF. The computing unit CU can in particular comprise a plurality of sub-computing units that execute different steps of the respective methods. In other words, the computing unit CU can also be understood to be a large number of computing units CU.

The following formulations and exemplary embodiments are also part of the disclosure. In particular, the determination system can also be embodied analogously to the methods described here:

A computer-implemented method for determining a similarity measure, wherein the similarity measure describes a similarity between a first patient PAT.1 and a second patient PAT.2, comprising:
receiving REC-PD.1 a first patient data record PD.1, wherein the first patient data record PD.1 is assigned to the first patient PAT.1;
receiving REC-PD.2 a second patient data record PD.2, wherein the second patient data record PD.2 is assigned to the second patient PAT.2;
receiving or determining REC-DET-ONT.M a medical ontology ONT.M, wherein the medical ontology ONT.M is independent of the first patient data record PD.1 and the second patient data record PD.2;
determining DET-ONT.P1 a first patient ontology ONT.P1 based on the medical ontology ONT.M and the first patient data record PD.1;
determining DET-ONT.P2 a second patient ontology ONT.P2 based on the medical ontology ONT.M and the second patient data record PD.2,
determining DET-SV the similarity measure based on the first patient ontology ONT.P1 and the second patient ontology ONT.P2.

A computer-implemented method for determining a similarity measure, wherein the similarity measure describes a similarity between a first patient PAT.1 and a second patient PAT.2, comprising:
receiving REC-PD.1 a first patient data record PD.1, wherein the first patient data record PD.1 is assigned to the first patient PAT.1;
receiving REC-PD.2 a second patient data record PD.2, wherein the second patient data record PD.2 is assigned to the second patient PAT.2;
receiving or determining REC-DET-ONT.M a medical ontology ONT.M, wherein the medical ontology ONT.M is independent of the first patient data record PD.1 and the second patient data record PD.2;
determining DET-ONT.CP a common patient ontology ONT.CP based on the medical ontology ONT.M, the first patient data record PD.1 and the second patient data record PD.2;
determining DET-SV the similarity measure based on the common patient ontology ONT.CP.

A computer-implemented method for determining a similarity measure, wherein the similarity measure describes a similarity between a first patient PAT.1 and a second patient PAT.2, comprising:
receiving REC-PD.1 a first patient data record PD.1, wherein the first patient data record PD.1 is assigned to the first patient PAT.1;
receiving REC-PD.2 of a second patient data record PD.2, wherein the second patient data record PD.2 is assigned to the second patient PAT.2;
receiving or determining REC-DET-ONT.M a medical ontology ONT.M, wherein the medical ontology ONT.M is independent of the first patient data record PD.1 and the second patient data record PD.2;
determining DET-ONT.CP a common patient ontology ONT.CP based on the medical ontology ONT.M, the first patient data record PD.1 and the second patient data record PD.2;
determining DET-ONT.P1 a first patient ontology ONT.P1 based on the common patient ontology ONT.CP;
determining DET-ONT.P2 a second patient ontology ONT.P2 based on the common patient ontology ONT.CP;
determining DET-SV the similarity measure based on the first patient ontology ONT.P1 and the second patient ontology ONT.P2.

Where not explicitly described, but advisable and within the spirit of the invention, individual exemplary embodiments, individual partial aspects or features thereof can be combined with or replaced by one another without departing from the scope of the present invention. Where applicable, advantages of the invention described with respect to one exemplary embodiment also apply without specific mention to other exemplary embodiments.

1. A computer-implemented method for determining a similarity measure, wherein the similarity measure describes a similarity between a first patient and a second patient, comprising:
receiving a first patient data record, wherein the first patient data record is assigned to the first patient;
receiving a second patient data record, wherein the second patient data record is assigned to the second patient;
receiving or determining a medical ontology, wherein the medical ontology is independent of the first patient data record and the second patient data record;
determining a patient ontology based on the medical ontology and at least one of the first patient data record or the second patient data record; and
determining the similarity measure based on the patient ontology.

2. The method of claim 1, wherein
the patient ontology is a common patient ontology, and
the common patient ontology is based on the medical ontology, the first patient data record and the second patient data record.

3. The method of claim 2, wherein
the common patient ontology comprises a graph, wherein a subgraph relates to the medical ontology,
the first patient data relates to at least one first node outside the subgraph and at least one edge between the first node and the subgraph,
the second patient data relates to at least one second node outside the subgraph and at least one edge between the second node and the subgraph, and
the similarity measure is based on a probability of an edge between the first node and the second node.

4. The method of claim 2, further comprising:
determining a first patient ontology based on the common patient ontology; and
determining a second patient ontology based on the common patient ontology,
wherein the determining the similarity measure is based on the first patient ontology the second patient ontology.

5. The method of claim 1, wherein the patient ontology is a first patient ontology and
the first patient ontology is not based on the second patient data record, the method further comprising:
determining a second patient ontology based on the medical ontology and the second patient data record, wherein the determining the similarity measure is based on the first patient ontology and the second patient ontology.

6. The method of claim 5, wherein
the first patient ontology comprises a first graph,
the second patient ontology comprises a second graph, and
the similarity measure is based on a similarity between the first graph and the second graph.

7. The method of claim 6, wherein the similarity measure comprises at least one of the following measures:
a graph edit distance of the first graph and the second graph, or
a maximum common subgraph distance of the first graph and the second graph.

8. The method of claim 6, wherein the similarity measure is based on at least one of vertex embedding or graph embedding of the first graph and the second graph.

9. The method of claim 1, wherein
the determining the similarity measure is based on an application of a trained function to at least one of the first patient ontology or the second patient ontology, or
the determining the similarity measure is based on an application of a trained function to the common patient ontology.

10. The method of claim 1, wherein the similarity measure includes a plurality of similarity measures for a plurality of second patient data records and the second patient data records are assigned to a plurality of second patients, the method further comprising:
determining a set of comparable patients based on the determined similarity measures, wherein the set of comparable patients is a subset of the plurality of second patients, and each of the comparable patients is similar to the first patient.

11. The method of claim 10, further comprising:
determining a probability value for a side effect of medical treatment of the first patient based on the set of comparable patients.

12. The method of claim 10, further comprising:
determining a probability value for an outcome of medical treatment of the first patient based on the set of comparable patients.

13. The method of claim 1, wherein the patient ontology is based on at least one of the following types of data:
genomic data from at least one of the first patient data record or the second patient data record,
epigenomic data from at least one of the first patient data record or the second patient data record,
transcriptomic data from at least one of the first patient data record or the second patient data record,
proteomic data from at least one of the first patient data record or the second patient data record, or
metabolomic data from at least one of the first patient data record or the second patient data record.

14. The method of one of the preceding claims, wherein the medical ontology maps at least one of the following influences:
influence of a human genome on at least one of a human genome, an epigenome, a transcriptome, a proteome or a metabolome,
influence of a human epigenome on at least one of a human genome, an epigenome, a transcriptome, a proteome or a metabolome,
influence of a human transcriptome on at least of a human genome, an epigenome, a transcriptome, a proteome or a metabolome,
influence of a human proteome on at least of a human genome, an epigenome, a transcriptome, a proteome or a metabolome, or
influence of a human metabolome on at least one of a human genome, an epigenome, a transcriptome, a proteome or a metabolome.

15. The method of claim 1, wherein at least one of
the patient ontology is based on one of the following types of data,
at least one of a genome sequence, germline mutations in the genome sequence or somatic mutations in the genome sequence of at least of one of the first patient or the second patient,
at least one of
pre-existing conditions or comorbidities of at least one of the first patient or the second patient,
symptoms occurring in at least one of the first patient or the second patient,
lifestyle of at least one of the first patient or the second patient, the lifestyle including at least one of alcohol consumption, tobacco consumption or drug consumption of at least one of the first patient or the second patient, or
physiological characteristics of at least one of the first patient or the second patient, the physiological characteristics including at least one of a height, a weight, an age, a gender or an ethnicity of at least one of the first patient or the second patient;
or the medical ontology is based on one of the following types of data,
a gene expression in a human organism,
at least one of
transcription factor binding sites, enhancer sites or splice sites with respect to at least one of a human genome or a transcriptome,
at least one of
amino acid sequences or protein domains with respect to a human proteome,
a spatial positional relationship of elements of a human genome or a proteome,
clinical annotations with respect to elements of a human a genome, an epigenome, a transcriptome, a proteome or a metabolome,
biological interaction pathways of a human genome, an epigenome, a transcriptome, a proteome or a metabolome, the biological interaction pathways including at least one of gene regulatory networks, metabolic pathways or signal transduction pathways,
interaction between conditions and symptoms in humans, or
interaction between pharmaceutical products, treatable diseases and side effects.

16. A determination system for determining a similarity measure, wherein the similarity measure describes a similarity between a first patient and a second patient, the determination system comprising:
an interface configured to,
receive a first patient data record, wherein the first patient data record is assigned to the first patient,
receive a second patient data record, wherein the second patient data record is assigned to the second patient; and
a computing unit,
wherein the interface or the computing unit is configured to receive or determine a medical ontology, and the medical ontology is independent of the first patient data record and the second patient data record, the computing unit being configured to,
determine a patient ontology based on the medical ontology and at least one of the first patient data record or the second patient data record, and
determine the similarity measure based on the patient ontology.

17. A non-transitory computer program product including program sections that, when executed by a determination system, cause the determination system to perform the method of claim 1.

18. A non-transitory computer-readable storage medium including program sections that, when executed by a determination system, cause the determination system to perform the method of claim 1.

19. The method of claim 8, wherein
the determining the similarity measure is based on an application of a trained function to at least one of the first patient ontology or the second patient ontology, or
the determining the similarity measure is based on an application of a trained function to the common patient ontology.

20. The method of claim 19, wherein the similarity measure includes a plurality of similarity measures for a plurality of second patient data records and the second patient data records are assigned to a plurality of second patients, the method further comprising:
determining a set of comparable patients based on the determined similarity measures, wherein the set of comparable patients is a subset of the plurality of second patients, and each of the comparable patients is similar to the first patient.
